(19) United States
(12) Patent Application Publication
(10) Pub. No.: US 2014/0348821 A1
(43) Pub. Date: Nov. 27, 2014

(54) Defective Mismatch Repair and Benefit from Bevacizumab for Colon Cancer

(71) Applicant: NSABP Foundation, Inc., Pittsburgh, PA (US)

(72) Inventors: Katherine Lea Pogue-Geile, Pittsburgh, PA (US); Soonmyung Paik, Pittsburgh, PA (US)

(73) Assignee: NSABP Foundation, Inc., Pittsburgh, PA (US)

(21) Appl. No.: 14/286,796

(22) Filed: May 23, 2014

Related U.S. Application Data
(60) Provisional application No. 61/827,435, filed on May 24, 2013.

(51) Int. Cl. C07K 16/22; A61K 45/06; A61K 39/395; C12Q 1/68; G01N 33/574

(52) U.S. Cl. CPC C07K 16/22; C12Q 1/6886; G01N 33/57419; A61K 39/39558; A61K 45/06; C12Q 2600/154; C12Q 2600/106; G01N 2333/922
USPC 424/133.1; 435/6.11; 435/7.4

(57) ABSTRACT

Methods of testing to identify and to treat a subset of colon cancer patients exhibiting dMMR tumor tissue, who derive significant clinical benefit from the addition of bevacizumab to standard adjuvant chemotherapy. The presence of a V600E BRAF mutation is also of significance.

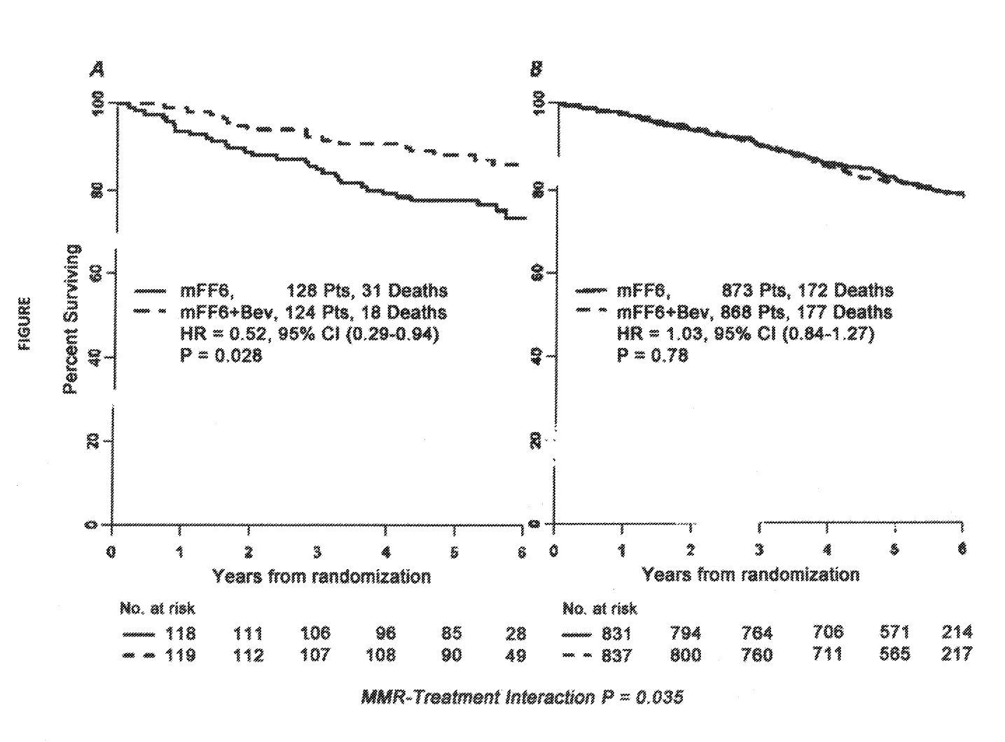

### CROSS REFERENCE TO RELATED APPLICATIONS

This application claims benefit of priority to U.S. Provisional Application No. 61/827435, file May 24, 2013, winch is incorporated herein by reference in its entirety.

Work described herein was supported by Public Health Service Grants U10-CA-37377, U10-CA-69974, U10-CA-12027, U10-CA-69651, and U24-CA-114732 from the National Cancer Institute, Department of Health and Human Services. The government has certain rights in the invention.

### BACKGROUND OF THE INVENTION

It is established in current medical best practices that for patients with Stage 0 and 1 and certain Stage II resectable colon cancer adjuvant therapy is not required according to the NCCN Clinical Practice Guidelines in Oncology. (http://bit.ly/lBoBlF). High and intermediate risk Stage II as well as Stage III and IV patients, however can benefit from regimens that include use of 5-FU and leucovorin with or without oxaliplatin or capecitabine. Preferred regimens are defined as (i) mFOLFOX6: oxialiplatin 85 mg/m² IV over 2 h on day 1 plus leucovorin 400 mg/m² IV over 2 h on day 1 plus 5-FU 400 mg/m² IV bolus on day 1, then 1200 mg/m²/day for 2d continuous infusion; repeat every 2wk; (ii) FLOX: 5-FU 500 mg/m² IV weekly plus leucovorin 500 mg/m² IV weekly for 6 wk (days 1, 8, 15, 22, 29, and 36) of each 8-wk cycle plus oxaliplatin 85 mg/m² IV administered on days 1, 15, and 29 of each 8-wk cycle for 3 cycles; (iii) Capecitabine 1250 mg/m² PO BID on days 1-14; repeat cycle every 21 d for 8 cycles; (iv) CapeOx; Oxaliplatin 130 mg/m² over non day 1 plus capecitabine 1000 mg/m² PO BID on days 1-14 every 3 wk for 8 cycles; (v) Leucovorin 500 mg/m² given as a 2-h infusion and repeated weekly for 6 wk plus 5-FU 500 mg/m² given. as a bolus 1 h after the start of leucovorin and repeated 6 times weekly; every 8 wk for 4 cycles; or Leucovorin 400 mg/m² IV over 2 h on day 1 plus 5-FU bolus 400 mg/m², then 1200 mg/m²/day for 2 d (total 2400 mg/m² over 46-48 h) continuous infusion; repeat every 2 wk.

Bevacizumab can also be administered concomitantly with various regimens as follows: (i) mFOLFOX6 plus bevacizumab 5 mg/kg over 30-90 min on day 1; (ii) FLOX plus bevacizumab 5 mg/kg over 30-90 min on days 1, 15, and 29; (iii) FOLFIRI plus bevacizumab 5 mg/kg over 30-90 min on day 1; (iv) CAPEOX plus bevacizumab 7.5 mg/kg over 30-90min on day 1; or (v) Capecitabine plus bevacizumab 7.5 mg/kg on day 1; for example.

### SUMMARY OF INVENTION

The present disclosure arises from a National Surgical Adjuvant Breast and Bowel Project protocol C-08 test of the worth of adding one year of bevacizumab oxaliplatin-based standard adjuvant chemotherapy regimen in the treatment of stage II/III colon cancer, While the overall result was negative, it was contemplated by the inventors that a molecularly defined subset could benefit from bevacizumab. Post-hoc statistical tests for marker-by-treatment interactions were performed for standard pathological features and it was found that patients diagnosed with mismatch repair defective (dMMR) tumors derived significant survival benefit from the addition of bevacizumab (hazard ratio=0.52 for overall survival) in contrast to no benefit in patients diagnosed with mismatch repair proficient (pMMR) tumors (hazard ratio=1.03) with an interaction p-value of 0.035. The inventions disclosed herein, therefore include methods of diagnosis and treatment of a molecularly defined subset of colon cancer that unexpectedly derives clinical benefit from anti-angiogenesis agents like bevacizumab.

### BRIEF DESCRIPTION OF THE DRAWING

The following drawing forms part of the present specification and is included to further demonstrate certain aspects of the present invention. The invention may be better understood by reference to this drawing in combination with the detailed description of specific embodiments presented herein.

The FIGURE is a graphical depiction of the effect of bevacizumab treatment on overall survival by MMR status for colon cancer in which Panel A is MMR Deficient and Panel B is MMR Proficient. In each panel the survival estimates are derived by the Kaplan-Meier method and the hazard ratio (HR), confident intervals (CIs) and P value come from a Cox regression model containing only an indicator variable for treatment. The MMR treatment interaction test (P=0.035) is from a Cox regression test. The model includes variables for MMR, bevacizumab treatment, and the interaction term. All statistical tests were two sided.

### DETAILED DESCRIPTION

While the anti-VEGF antibody bevacizumab showed promise for the treatment of stage IV colon cancer. (Hurwitz et al., N Engl J Med 2004;350(23):2335-42; Hurwitz et al., J Clin Oncol 2005;23(15):3502-8; Kabbinavar et at., Oncol 2003;21(1):60-5; Kabbinavar et al., J Clin Oncol 2005;23(16):3706-12) it failed to improve clinical outcome of patients diagnosed with stage II/III colon cancer when added to adjuvant chemotherapy. The C-08 protocol conducted by the National Surgical Adjuvant Breast and Bowel Project (NSABP) randomly assigned 2,710 patients diagnosed with stage II/III colon adenocarcinoma to receive either oxaliplatin-based Chemotherapy (mFOLFOX6) or mFOLFOX6 plus bevacizumab for 12 months (Allegra et. al. J Clin Oncol. 2011;29(1)11-6.) According to the primary end point analysis after median follow up of 35.6 months, the addition of bevacizumab to mFOLFOX6 did not result in a significant increase in disease free survival (HR-0.89; CI, 0.76 to 1.04; p=0.15). Tests for a potential interaction of the effect of bevacizumab with sex, age, and nodal status were not statistically significant. However, mismatch repair status (MMR) was not examined at that time.

The inventors have updated the analysis of C-08 with the inclusion of MMR status and longer know up. MMR status was determined by immunohistochernistry (IHC) with MLH1 and MSH2 proteins as described by Lindor et al., (J Clin Oncol 2002;20(4):1043-8). Any cases that showed negative staining of one of the two proteins in the tumor cells in the presence of positive staining in the surrounding normal cells were classified as MMR deficient (dMMR) while others were classified as MMR proficient (pMMR). These two IHC markers provide both a sensitive and specific alternative to microsatellite instability in detecting DNA MMR detects (Lindor et. al., J Clin Oncol 2002;20(4):1.043-8), The C-08 correlative study was conducted with approvals from institutional review boards for NSABP Biospecimen Bank and Biostatistics Center. Informed consent was required for participation. Formalin-fixed paraffin-embedded tumor blocks were available from 2100 of 2710 randomized patients. Patient characteristics of the MMR study subset were not different from the original trial cohort (Table 1). MMR status could be determined in 1993 cases. There were 107 cases with either assay failures with no staining in the normal cells or tissue detachment during the staining procedure. There were 252 cases (12.6%) classified as dMMR. In the set of patients with known MMR status, 25% were stage II and median follow-up was 5.7 years (range 0.2 to 7.4 years).

The V600E BRAF mutation was also examined based an its association with dMMR and worse overall survival (OS) (Gavin et al., Clinical Cancer Research 2012; December 1;18(23):6531-41 .) V600E mutation was determined using a primer extension assay as reported by Fumagalli, (N=1764) (Fumagalli et al., BMC Cancer 2010;10:101.)

Formal statistical tests for marker-by-bevacizumab interaction were performed for the following variables: age (<65 versus≧65, N=2159), gender (N-2159), T stage (N=2145), N stage (N-2159), MMR defects defined by two the markers (MLH1 and MSH2) (N=1993), and V600E BRAF mutation (N-1764) (Table 1). For the OS endpoint, only MMR status showed significant interaction with bevacizumab (P-0.0345) with a decrease in mortality observed only in patients with dMMR tumors. While 31 of 128 patients with dMMR tumors treated with chemotherapy died, only 18 of 124 patients who received bevacizumab in addition to chemotherapy died during the same follow-up period (HR=0.52, 95% CI: 0.29-0.94, p=0.028) (Figure Panel A). In contrast there was no difference in mortality between the control arm and bevacizumab arm in those who were diagnosed with pMMR tumors. There were 172 of 873 pMMR patients treated with chemotherapy who died whereas 177 of 868 pMMR patients treated with bevacizumab died during the same follow-up period (HR=1.03, 95% CI: 0.54-1.27, p=0.78) (Figure Panel B). For time-to-recurrence there was a trend for interaction in the same direction but it was not statistically significant (p-value for interaction 0.0819).

Although BRAF did not show significant interaction, since there was an association between MMR status and BRAF mutation (p<0.0001), we examined whether a combination of the two markers could further define the subset that benefited from bevacizumab in an exploratory analysis. We found that a small subset of patients (N=51 with 16 deaths), defined by BRAF mutation and dMMR derived the most benefit with a HR of 0.27 (95% CI 0.08-0.94, p=0.028),

Because dMMR was defined based on two IHC markets (MLH1 and MSH2), it is contemplated that about 25% of hyper-mutated tumors (with mutations in MLH3, MSH3, MSH6, PMS2, and POLE) could have been misclassified as pMMR based on data from The Cancer Genome Atlas Network (TCGA) (Nature 2012;487(7407):330-7). It is contemplated that patients diagnosed with hyper-mutated tumors due to the mutations in the latter genes also derive significant clinical benefit from bevacizumab.

According to published exome capture sequencing data from The Cancer Genome Atlas, dMMR tumors are hypermutated with a median number of non-silent mutations of 728 compared to 58 in pMMR or non-hypermutated tumors (The Cancer Genome Atlas Network. Nature 2012;487(7407)330-7). Unlike pMMR tumors that are poorly immunogenic, dMMR tumors are highly immunogenic due to the generation of mutated proteins including those with frame-shift mutations (Saeterdal et al., Proc Natl Acad Sci U S A 2001;98(23):13255-60; Banejea et al., Colorectal Dis 2009; 11(6):601-8). Therefore, dMMR tumor cells at the micro-metastatic sites have to evade attack from the immune system in order to progress. VEGF-A is speculated to he one of the main tumor-derived soluble factors that act as a chemo-attractant for immature myeloid cells from the marrow to the tumor site and suppresses dendritic cell maturation, creating an immune suppressive microenvironment Bellamy et al., Blood 2001;97(5):1427-34; Gabrilovich et al., Nat Med 1996;2(10):1096-103; Ohm et al., Blood 2003;101(12):4878-86; Oyama et al., J immunol 1998;1601(3):1224-32). Furthermore, VEGF-A directly induces regulatory T-cell (Treg) proliferation in tumor-bearing mice through VEGFR-2 (Terme et al., Cancer Res 2013;73:539-49). Intriguingly, blocking VEGF-A alone was sufficient to inhibit Treg cell accumulation in tumor-bearing mice but not in tumor-naïve mice (Terme et al., Cancer Res 2013;73:539-49). More importantly, adding bevacizumab to chemotherapy resulted in a significant reduction in the proportion of Treg cells in the peripheral blood of colon cancer patients (Terme et al., Cancer Res 2013;73:539-49). Without limiting the present disclosure to any particular theory, it is thus contemplated that bevacizumab is particularly effective in dMMR patients due to its disruption of the immunosuppressive microenvironment associated with these hypermutated and highly immunogenic tumors.

TABLE 1
Patient Characteristics: NSABP C-08

| | Trial Eligible | | Study Subset | | |
|---|---|---|---|---|---|
| | N = 2673 | | N = 2159 | | |
| Characteristic | N | % | N | % | P-value* |
| Age | | | | | 0.97 |
| <50 | 674 | 25.2 | 538 | 24.9 | |
| 50-59 | 882 | 33.0 | 714 | 33.1 | |
| 60-69 | 713 | 26.7 | 584 | 27.0 | |
| 70+ | 404 | 15.1 | 323 | 15.0 | |
| Sex | | | | | 0.47 |
| Female | 1342 | 50.2 | 1067 | 49.4 | |
| Male | 1331 | 49.8 | 1092 | 50.6 | |
| Race | | | | | 0.86 |
| White | 2333 | 87.3 | 1897 | 87.9 | |
| Black | 215 | 8.0 | 170 | 7.9 | |
| Other | 93 | 3.5 | 69 | 3.2 | |
| Muiti-racial | 3 | 0.1 | 3 | 0.1 | |
| Unknown | 29 | 1.1 | 20 | 0.9 | |
| ECOG Performance | | | | | 0.49 |
| 0 Fully active | 2164 | 81.0 | 1736 | 80.4 | |
| 1 No strenuous activity | 508 | 19.0 | 423 | 19.6 | |
| Nodal Stage | | | | | 0.92 |
| N0 (node negative) | 666 | 24.9 | 530 | 24.5 | |
| N1 (1-3 pos. nodes) | 1218 | 45.6 | 990 | 45.9 | |
| N2 (4+ pos. nodes) | 789 | 29.5 | 639 | 29.6 | |
| Treatment | | | | | 0.69 |
| mFOLFOX6 | 1338 | 50.1 | 1090 | 50.5 | |
| mFOLFOX6 + Bev | 1335 | 49.9 | 1069 | 49.5 | |

*Pearson Chi Squared test of whether the study subset is a representative sample of the trial-eligible patients.

TABLE 2
Variables examined and their interaction with bevacizumab

| | | Time to Recurrence | | Overall Survival | |
|---|---|---|---|---|---|
| | | | Marker-by- | | Marker by- |
| | | | bevacizumab | | bevacizumab |
| | | Recurrences | interaction | Deaths | interaction |
| Variable | N | N (%) | P* | N (%) | P* |

Age

| | | | | | |
|---|---|---|---|---|---|
| <65 | 1556 | 342 (21.0) | .4387 | 272 (17.5) | .4170 |
| ≧65 | 603 | 129 (21.4) | | 161 (26.7) | |

Gender

| | | | | | |
|---|---|---|---|---|---|
| Female | 1067 | 224 (21.0) | .2889 | 192 (18.0) | .4200 |
| Male | 1092 | 247 (22.6) | | 241 (22.1) | |

T-stage†

| | | | | | |
|---|---|---|---|---|---|
| Low | 713 | 66 (9.3) | .4984 | 77 (10.8) | .5125 |
| High | 1432 | 403 (28.1) | | 354 (24.7) | |

N stage

| | | | | | |
|---|---|---|---|---|---|
| N0 | 530 | 45 (8.5) | .2543 | 53 (10.0) | .2090 |
| N1 | 990 | 178 (18.0) | | 166 (16.8) | |
| N2 | 639 | 248 (38.8) | | 214 (33.5) | |

MMR Status

| | | | | | |
|---|---|---|---|---|---|
| Proficient | 1741 | 394 (22.6) | .0819 | 349 (20.1) | .0345 |
| Deficient | 252 | 32 (12.7) | | 49 (19.4) | |

BRAF

| | | | | | |
|---|---|---|---|---|---|
| Not | 1563 | 352 (22.5) | .2821 | 307 (19.6) | .3743 |
| mutated | | | | | |
| Mutated | 201 | 43 (21.4) | | 54 (26.9) | |

P* is for the interaction in a Cox model containing bevacizumab, the variable, and the variable-bevacizumab interaction.
†stage category is defined as “low” for Stage II T3 and Stage III T1 & T2 and “high” for Stage II T4 and Stage III T3 & T4.

TABLE 3
MMR deficient tumors are associated with BRAF mutations

| | | | Mismatch Repair Status | | |
|---|---|---|---|---|---|
| | | Total | Unknown | Proficient | Deficient |
| BRAF | WT (n) | 1910 | 422 | 1358 | 130 |
| | Mutant (n) | 316 | 69 | 176 | 71 |
| | % | 14.20% | | 11.50% | 35.30% |

| | |
|---|---|
| ρ | <0.0001 |

| | | |
|---|---|---|
| MMR | Unknown | 503 |
| Status | Stable | 1589 |
| | Unstable | 207 |
| | % | 11.50% |
| | ρ | |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. All such similar substitutes and modifications apparent to those skilled in the an are deemed to he within the spirit, scope and concept of the invention as defined by the appended claims.

1. A method for treating a cancer patient comprising:
identifying a cancer patient with mismatch repair deficient (dMMR) tumor tissue; and administering an anti-angiogenesis agent to the patient.

2. The method of claim 1, wherein the patient is a colon cancer patient.

3. The method of claim 1, wherein the patient is a Stage II or III colon cancer patient.

4. The method of claim 1, wherein the mismatch repair deficient tissue comprises a mutation in one of more of MLH1, MSH2, MLH3, MSH3, MSH6, PMS2, and POLE.

5. The method of claim 4, wherein the mutation comprises one or more mutations that render the protein non-functional.

6. The method of claim 1, wherein the tumor tissue further comprises a V600E BRAF mutation.

7. The method of claim 1, wherein the tumor tissue further comprises a microsatellite instability phenotype (MSI).

8. The method of claim 1, wherein the tumor tissue further comprises a CpG island methylator phenotype (CIMP) high phenotype.

9. The method of claim 1, wherein the anti-angiogenesis agent is an agent that blocks Vascular Endothelial Growth Factor (VEGF), its receptor, or its signaling pathway.

10. The method of claim 1, wherein the anti-angiogenesis agent is bevacizumab.

11. The method of claim 10, wherein the bevacizumab is administered concomitantly with an additional systemic chemotherapy agent.

12. The method of claim 11, wherein the additional systemic chemotherapy comprises administration of an agent selected from 5-fluorouracil (5-FU), leucovorin, oxaliplatin, capecitabine, irinotecan, and combinations thereof.

13. The method of claim 11, wherein bevacizumab is administered concomitantly with a regimen selected from mFOLFOX6, FLOX, FOLFIRI, CAPEOX or Capecitabine.

14. The method of claim 11, wherein the additional systemic chemotherapy agent comprises oxaliplatin-based chemotherapy.

15. The method of claim 14, wherein the additional systemic chemotherapy comprises Oxaliplatin 85 mg/m² IV over 2 h on day 1 plus leucovorin 400 mg/m² IV over 2 h on day 1 plus 5-FU 400 mg/m² IV bolus on day 1, then 1200 mg/m²/day for 2-d continuous infusion; (mFOLFOX6).

16. A method of identifying a candidate for bevacizumab adjuvant cancer therapy comprising:
obtaining a sample of tumor tissue from a colon cancer patient; and
detecting methylation in the promoter region of MLH1 in the tumor sample,
wherein the detection of methylation in the MLH1 promoter region in the tumor sample identifies the patient as a candidate for bevacizumab adjuvant cancer therapy.

17. A method of identifying a candidate for bevacizumab adjuvant cancer therapy comprising
obtaining a sample of tumor tissue from a colon cancer patient; and
detecting a CIMP high/mismatch repair deficient phenotype in the tumor tissue;
wherein the detection of the CIMP high/mismatch repair deficient phenotype in the tumor sample identifies the patient as a candidate for bevacizumab adjuvant cancer therapy.

18. A method of identifying a candidate far bevacizumab adjuvant cancer therapy comprising:
obtaining a sample of tumor tissue from a colon cancer patient; and
detecting hypermethylation in the tumor tissue;
wherein the detection of hypermethylation in the tumor sample identities the patient as a candidate tor bevacizumab adjuvant cancer therapy.

19. A method of identifying a candidate for bevacizumab adjuvant cancer therapy comprising:
obtaining a sample of tumor tissue from a colon cancer patient;
contacting the sample with one or more antibodies to immunohistochemistry markers selected from MLH1, MSH-2, MLH3, MSH3, MSH6, PMS2, and POLE; and
identifying tumor samples as mismatch repair deficient when said tumor samples exhibit negative binding with one or more antibodies at the tumor sample and positive binding to the same antibodies in the surrounding normal tissue far one or more of said immunohistochemistry markers;
wherein a colon cancer patient with as tumor tissue sample identified as mismatch repair deficient is identified as a candidate for bevacizumab adjuvant therapy.

20. The method of claim 19, wherein the patient is a Stage II or III colon cancer patient.

21. The method of claim 20, further comprising detecting a V600E BRAF mutation in the tumor sample.

22. The method of claim 20, wherein the bevacizumab is administered concomitantly with an additional systemic chemotherapy agent.

23. The method of claim 22, wherein the additional systemic chemotherapy comprises administration of an agent selected from 5-fluorouracil (5-FU), leucovorin, oxaliplatin, capecitabine, irinotecan, and combinations thereof.

24. The method of claim 22, wherein bevacizumab is administered concomitantly with a regimen selected from m FOLFOX6, FLOX, FOLFIRI, CAPEOX or Capecitabine.

25. The method of claim 22, wherein the additional systemic chemotherapy agent comprises oxaliplatin-based chemotherapy.

26. The method of claim 25, wherein the additional systemic chemotherapy comprises oxaliplatin 85 mg/m² IV over 2 h on day 1 plus leucovorin 400 mg/m² IV over 2 h on day 1 plus 5-FU 400 mg/m² IV bolus on day 1, then 1200 mg/m²/day for 2-d continuous infusion; (mFOLFOX6).
